(19) United States
(12) Patent Application Publication
(10) Pub. No.: US 2017/0367411 A1
(43) Pub. Date: Dec. 28, 2017

(54) VAPORISER ASSEMBLY FOR AN AEROSOL-GENERATING SYSTEM

(71) Applicant: Fabien DUC, Carouge (CH)

(72) Inventor: Fabien DUC, Carouge (CH)

(21) Appl. No.: 15/630,117

(22) Filed: Jun. 22, 2017

Related U.S. Application Data
(63) Continuation of application No. PCT/EP2017/064045, filed on Jun. 8, 2017.

(30) Foreign Application Priority Data
Jun. 23, 2016 (EP) 16175303.3

(51) Int. Cl. A24F 47/00; H05B 3/00

(52) U.S. Cl. CPC A24F 47/008; H05B 3/0014; H05B 2203/021

(57) ABSTRACT

A vaporiser assembly for an aerosol-generating system may include a capillary element made from porous glass. The capillary element has a first end and a second end. The vaporiser assembly further comprises a heater element. The first end of the capillary element is configured to be fluidically connected to a liquid storage portion. The heater element is provided at the second end of the capillary element. The pore size of the capillary element is configured to allow a liquid aerosol-forming substrate from the liquid storage portion to be conveyed from the first end to the second end of the capillary element by capillary action. The capillary element has a pore size gradient such that an average pore size of the capillary element transitions or varies from larger pores at the first end of the capillary element to smaller pores at the second end of the capillary element.

### CROSS-REFERENCE TO RELATED APPLICATIONS

This is a continuation of and claims priority to PCT/EP2017/064045, filed on Jun. 8, 2017, and further claims priority to EP 16175303.3, filed on Jun. 23, 2016, both of which are hereby incorporated by reference in their entirety.

### BACKGROUND

### Field

Example embodiments relate to a vaporiser assembly for an aerosol-generating system and an aerosol-generating system with the vaporiser assembly.

### Description of Related Art

There are handheld electrically operated aerosol-generating systems that consist of a device portion comprising a battery and control electronics and a separate cartridge comprising a supply of liquid aerosol-forming substrate held in a liquid storage portion and an electrically operated vaporiser or heater element. The liquid storage portion may comprise a capillary material in which the liquid aerosol-forming substrate is absorbed. The capillary material is in contact with the heater element and ensures that the liquid is conveyed to the heater element, thereby allowing the generation of a vapor. The generated vapor subsequently cools to form an aerosol. The capillary material may have a fibrous or spongy structure. The capillary material may be a porous material conveying the liquid from the liquid storage portion to the heater element. The capillary material and the heater element are provided, together with the liquid storage portion, in the cartridge. The cartridge is provided as a single-use cartridge, which is disposed once the liquid aerosol-forming substrate held in the liquid storage portion is depleted. The capillary material and the heater element are therefore disposed together with the cartridge, and a new capillary material and a new heater element are required for each new cartridge. Furthermore, unwanted burning residues can develop on a surface of the capillary material during an operation of the system.

### SUMMARY

A vaporiser assembly for an aerosol-generating system may comprise a capillary element and a heater element. The capillary element may be made of porous glass. The capillary element has a first end and a second end. The first end of the capillary element is configured to be fluidically connected to a liquid storage portion containing a liquid aerosol-forming substrate. A pore size of the capillary element is configured to allow the liquid aerosol-forming substrate from the liquid storage portion to be conveyed from the first end of the capillary element to the second end of the capillary element by capillary action. The capillary element has a pore size gradient such that an average pore size of the capillary element transitions from larger pores at the first end of the capillary element to smaller pores at the second end of the capillary element. The heater element is disposed at the second end of the capillary element.

The capillary element may have a cylindrical shape with a first surface at the first end and a second surface at the second end, and the heater element may be disposed on the second surface of the capillary element.

The heater element may be provided at a circumferential surface of the capillary element adjacent to the second end of the capillary element.

The heater element may be an electric resistance heater.

The heater element may be structured as a metallic coating, a mesh heater, or a coil.

The pore size gradient of the capillary element may be linear.

A porosity of the smaller pores at the second end of the capillary element is configured to hinder a leakage of the liquid aerosol-forming substrate through the second end of the capillary element while allowing passage of an aerosol through the second end of the capillary element.

A size of the smaller pores at the second end of the capillary element may be between 0.3 and 250 microns.

In some example embodiments, a size of the smaller pores at the second end of the capillary element may be between 0.5 and 100 microns.

In some example embodiments, a size of the smaller pores at the second end of the capillary element is between 1 and 20 microns.

In some example embodiments, a size of the smaller pores at the second end of the capillary element is between 2 and 8 microns.

A size of the larger pores at the first end of the capillary element may be between 5 and 500 microns.

In some example embodiments, a size of the larger pores at the first end of the capillary element is between 10 and 250 microns.

In some example embodiments, a size of the larger pores at the first end of the capillary element is between 15 and 100 microns.

In some example embodiments, a size of the larger pores at the first end of the capillary element is between 20 and 50 microns.

An aerosol-generating system may comprise a main body including a housing, a power supply, electric circuitry, and a vaporiser assembly. The vaporiser assembly may include a capillary element and a heater element. The capillary element may be made of porous glass. The capillary element has a first end and a second end. The capillary element has a pore size gradient such that an average pore size of the capillary element transitions from larger pores at the first end of the capillary element to smaller pores at the second end of the capillary element. The heater element may be disposed at the second end of the capillary element.

The aerosol-generating system may further comprise a liquid storage portion detachably connected to the main body. The liquid storage portion contains a liquid aerosol-forming substrate. The liquid storage portion is configured to receive the first end of the capillary element of the vaporiser assembly such that the capillary element comes into fluidic communication with the liquid aerosol-forming substrate stored in the liquid storage portion. A pore size of the capillary element is configured to allow the liquid aerosol-forming substrate from the liquid storage portion to be conveyed from the first end of the capillary element to the second end of the capillary element by capillary action.

The aerosol-generating system may further comprise a sealing element disposed between a circumferential surface of the capillary element and the liquid storage portion to hinder a leakage of the liquid aerosol-forming substrate from the liquid storage portion.

A method for manufacturing a vaporiser assembly for an aerosol-generating system may comprise fabricating a capillary element from porous glass, the capillary element having a first end and a second end, the first end of the capillary element configured to be fluidically connected to a liquid storage portion containing a liquid aerosol-forming substrate, a pore size of the capillary element configured to allow the liquid aerosol-forming substrate from the liquid storage portion to be conveyed from the first end of the capillary element to the second end of the capillary element by capillary action, the capillary element having a pore size gradient such that an average pore size of the capillary element transitions from larger pores at the first end of the capillary element to smaller pores at the second end of the capillary element. The method further comprises providing a heater element at the second end of the capillary element.

The fabricating in accordance with the method of manufacturing may be performed with a phase separation process, a sintering process, or a sol-gel process.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a sectional view of a vaporiser assembly according to an example embodiment.

FIG. 2 is a sectional view of an aerosol-generating system according to an example embodiment.

### DETAILED DESCRIPTION

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, components, regions, layers and/or sections, these elements, components, regions, layers, and/or sections should not be limited by these terms. These terms are only used to distinguish one element, component, region, layer, or section from another region, layer, or section. Thus, a first element, component, region, layer, or section discussed below could be termed a second element, component, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments should not be construed as limited to the shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

According to some example embodiments, there is provided a vaporiser assembly for an aerosol-generating system. The vaporiser assembly may include a capillary element which comprises porous glass (e.g., formed of porous glass). The capillary element has a first end and a second end. The vaporiser assembly further comprises a heater element. The first end of the capillary element is configured to be fluidically connected to a liquid storage portion, and the heater element is provided at the second end of the capillary element. The pore size of the capillary element is configured to allow a liquid aerosol-forming substrate from the liquid storage portion to be conveyed from the first end of the capillary element to the second end of the capillary element by capillary action. The average pore size of the capillary element varies from larger pores at the first end of the capillary element to smaller pores at the second end of the capillary element such that a pore size gradient from the first end of the capillary element to the second end of the capillary element is provided.

Due to the fact that the capillary element can be made from glass, the heater element may be provided directly on the capillary element. In this regard, a benefit of the porous glass of the capillary element is that glass has an increased heat resistance. The capillary element is consequently not damaged or harmed by the increased temperature of the heating element during heating, even if the heater element is provided directly on the capillary element or in the near vicinity of the capillary element.

An increased heat resistance of the capillary element also leads to the effect that during heating of the liquid aerosol-forming substrate by the heater element, the risk of emitting undesirable products is reduced.

Also, since the capillary element can be made from porous glass, the capillary element may be more easily cleaned. The capillary element may be manually cleaned when the replaceable liquid storage portion is changed. The capillary element may also be cleaned during the insertion of the capillary element into a new liquid storage portion.

By providing the porous glass in the capillary element, the improved cleaning and heat resistance synergistically improve the reusability of the vaporiser assembly. Due to the improved heat resistance, unwanted residues on the capillary element and, thus, undesirable products are avoided or reduced during heating. The heater element may also be provided directly on the capillary element or in the near vicinity of the capillary element. At the same time, unwanted residues on the capillary element may be more easily cleaned.

Furthermore, glass is a relatively stable material, which does not degrade with temperature. Multiple replaceable liquid storage portions may therefore be used before the capillary element must be replaced.

Liquid storage portions may be used without the need to provide a new capillary element and a new heater element each time the liquid storage portion is replaced. The capillary element as well as the heater element is useable with multiple replaceable liquid storage portions. Therefore, the costs for the replaceable liquid storage portion are decreased.

The capillary element may have a cylindrical shape or form. Alternatively, the capillary element may have a different shape or form suited to be inserted into a replaceable liquid storage portion. The capillary element has a first surface at the first end and a second surface at the second end. The heater element is provided on the second end surface of the capillary element. The first surface and the second surface may have a round or ellipsoidal shape. Also, the first and second surface may have a polygonal shape (e.g., rectangular shape). Furthermore, the first surface and the second surface may be substantially flat or curved. A side surface is provided at the circumference of the capillary element (e.g., circumferential surface) between the first end and the second end. The first and second surface may have a diameter of between 1 millimeter and 15 millimeters. For instance, the diameter may be between 2 millimeters and 10 millimeters, between 3 millimeters and 7 millimeters, or between 4 millimeters and 6 millimeters (e.g., around 5 millimeters). The surface area of the first and second surface may be smaller than 60 square millimeters. For instance, the surface area may be smaller than 50 square millimeters, smaller than 40 square millimeters (e.g., around 30 square millimeters). The length of the capillary element may be between 1 millimeter and 7.5 centimeters. For instance, the length may be between 5 millimeters and 3 centimeters (e.g., around 1 centimeter). The liquid capacity of the capillary element is such that it can hold enough liquid aerosol-forming substrate for 30 to 40 puffs of more (e.g., around 32 puffs). A 3 second puff may include between 1 milligram and 4 milligrams of liquid (e.g., between 3 milligrams and 4 milligrams of liquid). The capacity of the capillary element may be between 30 milligrams and 160 milligrams. For instance, the capacity may be between 60 milligrams to 150 milligrams or between 90 milligrams to 140 milligrams (e.g., around 130 milligrams).

In an example embodiment, the capillary element is made from porous glass. The porous glass has an internal structure which allows liquids to be conveyed from the first end of the capillary element to the second end of the capillary element. In more detail, the porous glass comprises pores which enable liquid to travel through the capillary element.

In this regard, the pores, which are provided in the capillary element, enable the effect of intermolecular forces between the liquid aerosol-forming substrate and the surrounding glass material of the capillary element. The size (e.g., diameter) of the pores is configured such that the combination of surface tension and adhesive forces between the liquid aerosol-forming substrate and the surrounding glass material of the capillary element leads to the conveying of the liquid through the capillary element.

The term “porous” should be understood in a broad context or meaning. The pores of the capillary element are interconnected and may have a fibrous structure. The capillary element may comprise a bundle of capillaries. For example, the capillary element is manufactured by assembling and compressing glass particles similar to the manufacturing of ceramics. The size of the pores, which are generated during this process, depends on the applied force during the compression. The pore size may vary along the length of the cylinder. The pores may be generally aligned to convey the liquid aerosol-forming substrate to the heater element. The structure of the capillary element forms a plurality of relatively small pores, through which the liquid may be transported by capillary action. The capillary element may have any suitable capillarity and porosity so as to be used with different liquid physical properties. The liquid has physical properties, including but not limited to viscosity, surface tension, density, thermal conductivity, boiling point, and vapor pressure, which allow the liquid to be transported through the capillary element by capillary action.

The capillary element may comprise multiple materials, wherein one of these materials is the porous glass. The capillary element may also be entirely made of the porous glass. Also, multiple capillary elements could be provided next to each other, wherein one or more of the above capillary elements could be combined.

The capillary element may have a form that when the capillary element is inserted into a liquid storage portion, liquid present in the liquid storage portion cannot flow past the outer circumference of the capillary element. Consequently, liquid can only be conveyed out of the liquid storage portion through an end of the capillary element. A press-fit may be provided between the capillary element and the liquid storage portion, when the liquid storage portion is connected to the capillary element, such that liquid from the liquid storage portion may only flow out of the liquid storage portion through the capillary element. Liquid is prevented from flowing through the side surface of the capillary element by the liquid storage portion. In more detail, the press-fit between the capillary element and the liquid storage portion prevents the liquid flows through the side surface of the capillary element.

Alternatively, the pores, which are provided in the capillary element, are provided in a longitudinal direction between the first and second end of the capillary element such that liquid may only flow through the capillary element from the first end of the capillary element to the second end of the capillary element. The capillary element may comprise a fluid impermeable outer surface such as a fluid impermeable coating. The fluid impermeable coating may be applied to the outer surface of the capillary element to hinder or prevent leakage. Alternatively, the capillary element may be inserted into a fluid impermeable tube such as a glass tube. In the instance where the pores are provided in a longitudinal direction, the liquid cannot flow through the capillary element at the side surface of the capillary element, since the pores are not provided at the side surface of the capillary element.

As a further alternative, the pores at a side surface of the capillary element may be provided with a size which hinders or prevents liquid from leaking out of the side surface of the capillary element. For instance, the diameter or size of the pores, which are provided at the side surface of the capillary element, may be small enough that liquid cannot flow through these pores at the side surface of the capillary element.

The liquid may enter the capillary element at the first end of the capillary element and may be conveyed through the capillary element in the direction of the second end of the capillary element.

The average pore size of the pores which are provided in the porous glass varies or transitions from larger pores at the first end of the capillary element to smaller pores at the second end of the capillary element. A pore size gradient may be provided from the first end of the capillary element to the second end of the capillary element.

Smaller pores create a larger capillary force or action. Consequently, providing smaller pores at the second end of the capillary element ensures that the liquid aerosol-forming substrate from the liquid storage portion is drawn from the first end towards the second end of the capillary element. The pore size is configured to optimize or otherwise provide the desired flow rate. Smaller pores also hinder or prevent liquid from being leaked out of the capillary element while permitting vapor to flow through the smaller pores at the second end to enable a subsequent formation of an aerosol. The pore size of the smaller pores at the second may be between 0.3 and 250 microns, between 0.5 and 100 microns, between 1 and 20 microns, or between 2 and 8 microns (e.g., about 4 microns).

The average pore size of the pores at the first end of the capillary element is larger than the average pore size of the pores at the second end of the capillary element. The average pore size is an average pore size for a region of the capillary element. In this way, the liquid aerosol-forming substrate is conveyed more efficiently to the heater element. The pore size of the larger pores at the first end may be between 5 and 500 microns, between 10 and 250 microns, between 15 and 100 microns, or between 20 and 50 microns.

By providing a pore size gradient (e.g., a linear gradient) in the capillary element, the effect is achieved that an aerosol-forming substrate in the form of a liquid may be efficiently and in relatively large amounts conveyed from the liquid storage portion at the first end of the capillary element to the second end of the capillary element, which is adjacent to the heater element. The liquid may then be vaporised by the heater element next to the second end of the capillary element.

The heater element is provided at the second end of the capillary element such that liquid which is conveyed through the capillary element from the first end to the second end may be vaporised by the heater element. The heater element may be provided directly on the second end of the capillary element so that the heater element directly contacts the second end of the capillary element. Alternatively, the heater element may be provided in close proximity of the second end of the capillary element to heat the second end of the capillary element. The heater element may be provided at the circumference (e.g., circumferential surface) of the capillary element adjacent to the second end of the capillary element.

By providing the heater element at the circumference of the capillary element adjacent to the second end of the capillary element, a compact vaporiser assembly, comprising the capillary element and the heater element may be provided. Also, an efficient vaporisation of the liquid, which is conveyed from the first end of the capillary element to the second end of the capillary element, can be provided. By providing the heater element at the circumference of the capillary element adjacent to the second end of the capillary element, the capillary element can be more easily cleaned due to the second end surface of the capillary element not being blocked by the heating element.

In some example embodiments, the heater element is an electric resistance heater. The heater element comprises an electrically conductive material. The electrically conductive material may be heated by an electric current flowing through the electrically conductive material. The electrically conductive material may be provided on an electrically insulating substrate of the heater element.

The heater element may also comprise a glass material such that the capillary element and the heater element each comprise glass material. The electrically conductive material of the heater element may be provided in or on the heater element.

The electrical resistance of the heater element should be provided so that a sufficient heating of the aerosol-forming substrate at the second end surface of the capillary element is provided. In this regard, the electrical resistance of the electrically conductive material of the heater element may be between 2 ohms and 5 ohms. For instance, the electrical resistance may be between 3 ohms and 4 ohms (e.g., around 3.5 ohms).

In some example embodiments, the heater element may be provided as a metallic coating, thin film, a mesh heater, or a coil. When the heater element is provided as a metallic coating, the heater element may be provided directly on the second end surface of the capillary element. In more detail, the second end surface of the capillary element may be provided with an electrically conductive coating, which may be heated to vaporise liquid on the second end surface of the capillary element.

The heater element may also be provided as a mesh heater, which comprises multiple conductive filaments. This allows a greater area of the heater element to be in contact with a liquid being vaporised. The electrically conductive filaments may be substantially flat.

The heater element may be provided as a heater coil made from electrically conductive wire. A coil may be wound around the capillary element and is beneficial in case that the heater element is provided at the circumference of the capillary element adjacent to the second end of the capillary element.

Also provided is a vaporiser assembly for an aerosol-generating system. The vaporiser assembly may include a capillary element which comprises porous glass. The capillary element has a first end and a second end. The vaporiser assembly further comprises a heater element. The first end of the capillary element is configured to be fluidically connected to a liquid storage portion and the heater element is provided at the second end of the capillary element. The pore size of the capillary element is configured to allow a liquid aerosol-forming substrate from the liquid storage portion to be conveyed from the first end of the capillary element to the second end of the capillary element by capillary action.

According to some example embodiments, an aerosol-generating system is provided, which comprises a main body. The main body comprises a housing, a power supply, electric circuitry, and a vaporiser assembly as described in detail herein.

The aerosol-generating system may further comprise a replaceable or refillable liquid storage portion. The liquid storage portion is detachably connectable to the main body. When the liquid storage portion is attached to the main body, the first end of the capillary element of the vaporiser assembly is inserted into the liquid storage portion, such that the capillary element comes into fluidic communication with the liquid aerosol-forming substrate stored in the liquid storage portion.

The liquid storage portion may comprise a further capillary element. The further capillary element may be provided in the liquid storage portion. In this case, the glass capillary element of the vaporiser may be relatively thin to prevent the heater element from burning the further capillary element, which is provided in the liquid storage portion. The glass capillary element may have a thickness of at least 1 millimeter. For instance, the thickness may be at least 2 millimeters (e.g., at least 3 millimeters). The glass capillary element of the vaporiser assembly may still comprise small pores such that liquid aerosol-forming substrate does not leak through the capillary element, but vapor may flow through the capillary element for aerosol formation. The further capillary element may be provided from a porous material with a spongy or fibrous structure. The glass capillary element of the vaporiser assembly may be reusable, whereas the further capillary element may be disposed together with the liquid storage portion. Thus, the beneficial characteristics of glass can be utilized, while only a thin glass capillary element is in the vaporiser.

The power supply may be electrically connected to the heater element of the vaporiser assembly to enable heating of the heater element. The electric circuitry controls the flow of electric current from the power supply to the heater element. When the aerosol-generating device is actuated, the electric circuitry enables an electric current to flow from the power supply to the heater element of the vaporiser assembly, thereby vaporising an aerosol-forming substrate from a liquid storage portion and creating an aerosol. A sensor such as a flow sensor may be provided to detect the application of a negative pressure on the system.

In some example embodiments, a sealing foil may be provided on an opening of the liquid storage portion. During or before insertion of the capillary element into the liquid storage portion, the sealing foil may be removed. The aerosol-generating system may further comprise a sealing membrane, which is disposed beneath the sealing foil. When the sealing foil is removed and the capillary element is inserted into the liquid storage portion, the sealing membrane may be ruptured and pressed between the circumference (e.g., circumferential surface) of the capillary element and the replaceable liquid storage portion. The sealing membrane is provided to hinder or prevent an undesired leakage of the liquid aerosol-forming substrate out of the liquid storage portion. The liquid aerosol-forming substrate may flow through the capillary element but not past the outer circumference (e.g., exterior circumferential surface) of the capillary element, when the capillary element is inserted into the liquid storage portion.

According to some example embodiments, a method for manufacturing a vaporiser assembly for an aerosol-generating system is provided. The method comprises the steps of fabricating or providing a capillary element made from porous glass, the capillary element having a first end and a second end, and providing a heater element. The first end of the capillary element is configured to be fluidically connected to a liquid storage portion, wherein the heater element is provided at the second end of the capillary element. The pore size of the capillary element is provided to allow a liquid aerosol-forming substrate from the liquid storage portion to be conveyed from the first end of the capillary element to the second end of the capillary element by capillary action. The average pore size of the capillary element varies from larger pores at the first end of the capillary element to smaller pores at the second end of the capillary element such that a pore size gradient from the first end of the capillary element to the second end of the capillary element is provided.

In some example embodiments, the capillary element is fabricated or manufactured by a phase separation process, a sintering process, or a sol-gel process. These processes enable the capillary element to be provided with pores, which in turn enable a liquid aerosol-forming substrate to be conveyed through the capillary element. Furthermore, these processes enable the capillary element to be produced with pores of different size and with a pore size gradient from a first end of the capillary element to a second end of the capillary element.

It should be understood that features described in relation to one or more examples may equally be applied to other examples.

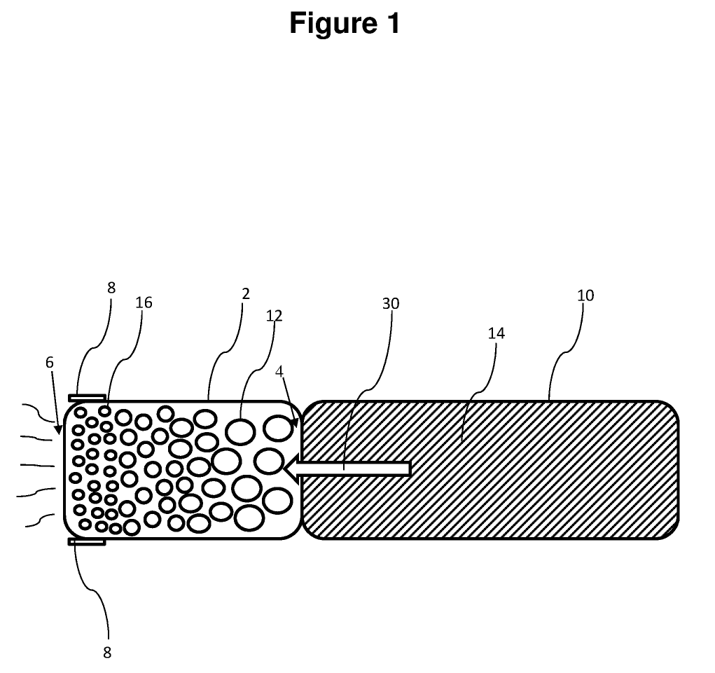

FIG. 1 shows a vaporiser assembly according to an example embodiment.

The vaporiser assembly comprises a capillary element 2 made from porous glass as depicted in the left part of FIG. 1. The capillary element 2 may comprise pores of varying sizes. The capillary element 2 has a first end 4 and a second end 6.

The porous glass of the vaporiser assembly is provided adjacent to a heater element 8. The heater element 8 as depicted in FIG. 1 is disposed at the circumference (e.g., circumferential surface) of the porous glass adjacent to the second end 6 of the porous glass. The first end 4 of the capillary element 2 faces a liquid storage portion 10.

At the first end 4 of the capillary element 2, large pores 12 with an average pore size of around 25 microns are provided as depicted in FIG. 1. The large pores 12 enable a liquid aerosol-forming substrate 14 to be conveyed from the liquid storage portion 10 in the direction of the second end 6 of the capillary element 2 through the capillary element 2. At the second end 6 of the capillary element 2, small pores 16 with an average pore size of around 4 microns are provided, thereby hindering or preventing liquid from leaking out of the second end 6 while enabling a flow or passage of a vapor through the second end 6.

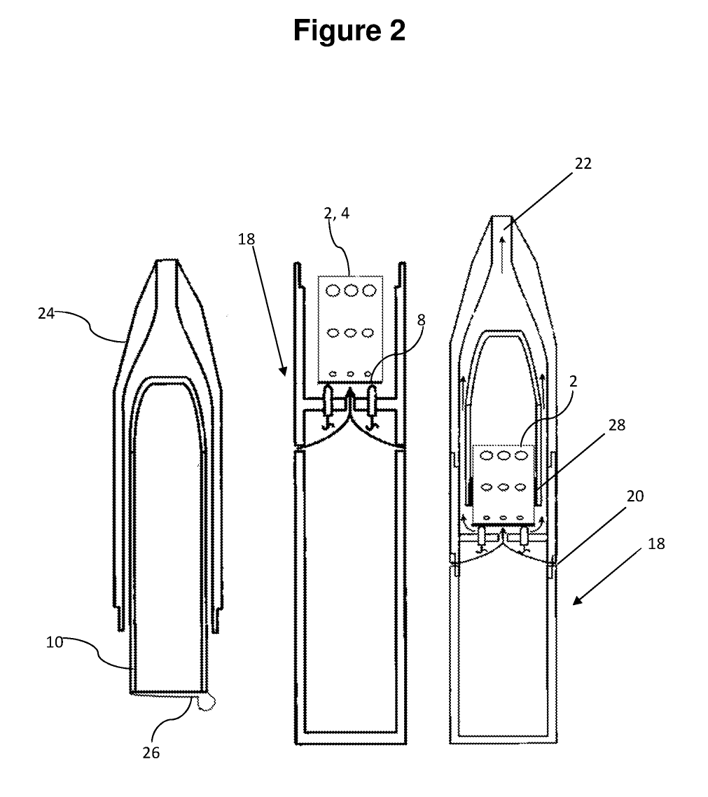

FIG. 2 shows an aerosol-generating system according to an example embodiment. Depicted in FIG. 2 is a main body 18 comprising the capillary element 2 of the vaporiser assembly, the heater element 8 as well as electrical circuitry and a power supply (not depicted in FIG. 2). The main body further comprises air inlets 20 and an air outlet 22. When a negative pressure is applied to a mouthpiece 24, ambient air is drawn through the air inlets 20 past the vaporiser assembly towards the air outlet 22. The electrical circuitry controls the flow of electric current from the power supply to the heater element 8 for heating the heater element 8. A flow sensor may detect when a negative pressure is applied to the mouthpiece 24. The electrical circuitry controls a flow of electric current from the power supply through the heater element. Consequently, the liquid aerosol-forming substrate 14 is vaporised by the heater element 8, thereby creating an aerosol.

The liquid storage portion 10 is provided with a sealing foil 26 which is provided on the liquid storage portion 10. The sealing foil 26 hinders or prevents the liquid aerosol-forming substrate 14 from leaking out of the liquid storage portion 10. The sealing foil 26 is removed before the liquid storage portion 10 is attached to the main body 18. Beneath the sealing foil 26, a sealing membrane 28 is provided to cover the liquid storage portion 10. The sealing membrane 28 is provided as a sealing element for sealing the outer circumference of the capillary element 2 when the capillary element 2 is inserted into the liquid storage portion 10 as described in the following paragraph.

When the liquid storage portion 10 is attached to the main body 18, the capillary element 2 of the vaporiser assembly is inserted into the liquid storage portion 10. The capillary element 2 is inserted into the liquid storage portion 10 such that the first end 4 of the capillary element 2 is inserted first into the liquid storage portion 10. During insertion of the capillary element 2 into the liquid storage portion 10, the sealing membrane 28 is ruptured and pressed against the inner wall of the liquid storage portion 10. The sealing membrane 28 may be provided with a desired or predetermined breaking area, a desired or predetermined breaking point, or an area with a deliberately placed weak point to localize and facilitate the rupture. Thus, the liquid aerosol-forming substrate 14 can only flow through the capillary element 2 to the second end 6, while the outer circumference of the capillary element 2 is sealed by the sealing membrane 28.

The liquid aerosol-forming substrate 14 is conveyed from within the liquid storage portion 10 through the first end 4 towards the second end 6 of the capillary element 2 of the vaporiser assembly in the direction 30 to the heater element 8 of the vaporiser assembly by capillary action.

While a number of example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

1. A vaporiser assembly for an aerosol-generating system, comprising:
a capillary element made of porous glass, the capillary element having a first end and a second end, the first end of the capillary element configured to be fluidically connected to a liquid storage portion containing a liquid aerosol-forming substrate, a pore size of the capillary element configured to allow the liquid aerosol-forming substrate from the liquid storage portion to be conveyed from the first end of the capillary element to the second end of the capillary element by capillary action, the capillary element having a pore size gradient such that an average pore size of the capillary element transitions from larger pores at the first end of the capillary element to smaller pores at the second end of the capillary element; and
a heater element disposed at the second end of the capillary element.

2. The vaporiser assembly according to claim 1, wherein the capillary element has a cylindrical shape with a first surface at the first end and a second surface at the second end, and the heater element is disposed on the second surface of the capillary element.

3. The vaporiser assembly according to claim 1, wherein the heater element is provided at a circumferential surface of the capillary element adjacent to the second end of the capillary element.

4. The vaporiser assembly according to claim 1, wherein the heater element is an electric resistance heater.

5. The vaporiser assembly according to claim 1, wherein the heater element is structured as a metallic coating, a mesh heater, or a coil.

6. The vaporiser assembly according to claim 1, wherein the pore size gradient of the capillary element is linear.

7. The vaporiser assembly according to claim 1, wherein a porosity of the smaller pores at the second end of the capillary element is configured to hinder a leakage of the liquid aerosol-forming substrate through the second end of the capillary element while allowing passage of an aerosol through the second end of the capillary element.

8. The vaporiser assembly according to claim 1, wherein a size of the smaller pores at the second end of the capillary element is between 0.3 and 250 microns.

9. The vaporiser assembly according to claim 1, wherein a size of the smaller pores at the second end of the capillary element is between 0.5 and 100 microns.

10. The vaporiser assembly according to claim 1, wherein a size of the smaller pores at the second end of the capillary element is between 1 and 20 microns.

11. The vaporiser assembly according to claim 1, wherein a size of the smaller pores at the second end of the capillary element is between 2 and 8 microns.

12. The vaporiser assembly according to claim 1, wherein a size of the larger pores at the first end of the capillary element is between 5 and 500 microns.

13. The vaporiser assembly according to claim 1, wherein a size of the larger pores at the first end of the capillary element is between 10 and 250 microns.

14. The vaporiser assembly according to claim 1, wherein a size of the larger pores at the first end of the capillary element is between 15 and 100 microns.

15. The vaporiser assembly according to claim 1, wherein a size of the larger pores at the first end of the capillary element is between 20 and 50 microns.

16. An aerosol-generating system, comprising:
a main body including a housing, a power supply, electric circuitry, and a vaporiser assembly, the vaporiser assembly including a capillary element and a heater element, the capillary element made of porous glass, the capillary element having a first end and a second end, the capillary element having a pore size gradient such that an average pore size of the capillary element transitions from larger pores at the first end of the capillary element to smaller pores at the second end of the capillary element, the heater element disposed at the second end of the capillary element.

17. The aerosol-generating system according to claim 16, further comprising:
a liquid storage portion detachably connected to the main body, the liquid storage portion containing a liquid aerosol-forming substrate, the liquid storage portion configured to receive the first end of the capillary element of the vaporiser assembly such that the capillary element comes into fluidic communication with the liquid aerosol-forming substrate stored in the liquid storage portion, a pore size of the capillary element configured to allow the liquid aerosol-forming substrate from the liquid storage portion to be conveyed from the first end of the capillary element to the second end of the capillary element by capillary action.

18. The aerosol-generating system according to claim 17, further comprising:
a sealing element disposed between a circumferential surface of the capillary element and the liquid storage portion to hinder a leakage of the liquid aerosol-forming substrate from the liquid storage portion.

19. A method for manufacturing a vaporiser assembly for an aerosol-generating system, comprising:
fabricating a capillary element from porous glass, the capillary element having a first end and a second end, the first end of the capillary element configured to be fluidically connected to a liquid storage portion containing a liquid aerosol-forming substrate, a pore size of the capillary element configured to allow the liquid aerosol-forming substrate from the liquid storage portion to be conveyed from the first end of the capillary element to the second end of the capillary element by capillary action, the capillary element having a pore size gradient such that an average pore size of the capillary element transitions from larger pores at the first end of the capillary element to smaller pores at the second end of the capillary element; and
providing a heater element at the second end of the capillary element.

20. The method according to claim 19, wherein the fabricating is performed with a phase separation process, a sintering process, or a sol-gel process.
